(19) United States
(12) Patent Application Publication
(10) Pub. No.: US 2003/0161806 A1
(43) Pub. Date: Aug. 28, 2003

(54) Care or makeup composition, comprising a non-volatile silicone compound, a non-silicone fat-soluble polymer and a specific dispersant

(76) Inventors: Pascal Arnaud, L'Hay Les Roses (FR); Vanina Filippi, Paris (FR); Xavier Blin, Paris (FR)

Correspondence Address: Thomas L. Irving, FINNEGAN, HENDERSON, FARABOW,, GARRETT & DUNNER, L.L.P., 1300 I Street, N.W., Washington, DC, 20005-3315, US

(21) Appl. No.: 10/337,786

(22) Filed: Jan. 8, 2003

Related U.S. Application Data
(60) Provisional application No. 60/346,980, filed on Jan. 11, 2002.

(30) Foreign Application Priority Data
Jan. 8, 2002 (FR) 02 00160

(51) Int. Cl. A61K 7/06; A61K 7/11

(52) U.S. Cl. 424/70.12

(57) ABSTRACT

An anhydrous care or makeup composition, which comprises, in a cosmetically acceptable medium, at least one non-volatile silicone compound, at least one non-silicone fat-soluble polymer and at least one hydrocarbon dispersant which has solubility parameters δd and δa which meet the following conditions: 16.2≦δ_{d}≦20 (J/cm³)^{1/2} and 9.1≦δₐ≦20 (J/cm³)^{1/2}, wherein the at least one hydrocarbon dispersant comprises carbon and hydrogen atoms and at least one functional group chosen from hydroxyl, ester, ether, carboxylic acid and amide functional groups and the composition can possess good properties of staying power, particularly as regards colour, and of gloss, comfort and non-migration; and the use thereof.

This application claims benefit of U.S. Provisional Application No. 60/346,980, filed Jan. 11, 2002.

Disclosed herein is an anhydrous cosmetic composition comprising a specific dispersant. This composition can possess notable cosmetic properties, such as staying power, and can give a makeup or care product properties of sheen, comfort and non-migration.

This composition may be provided, for example, in the form of a product cast in the form of a stick or dish such as lipsticks or lip balms, solid foundations, concealers, eyeshadows or rouges, in the form of a paste or of a fluid cream such as liquid foundations or lipsticks, eyeliners, mascaras, sun protection, skin colouring or artificial tanning compositions and hair or body makeup compositions.

The products for making up or caring for the skin or lips of human beings, such as foundations or lipsticks, generally comprise fatty phases such as waxes and oils, pigments and/or fillers and, optionally, additives such as cosmetically active substances. They may also include what are termed “pastelike” products, of flexible consistency, which make it possible to obtain coloured or uncoloured pastes intended for application by brush.

The known compositions, such as makeup compositions, have a tendency to migrate: they tend to spread over time inside the folds, wrinkles and lines of the skin which surround in particular the lips and eyes, giving rise to an unattractive effect. This migration is frequently mentioned by women as being a major defect of conventional lipsticks and eyeshadows. The term “migration” means the running of the composition and, for example, the running of the colour, beyond the initial outline of the makeup.

Furthermore, these compositions can exhibit poor staying power over time, such as with regard to the colour. This poor staying power is manifested in an alteration in colour (colour change, fade) generally as a result of interaction with the sebum and/or perspiration secreted by the skin, in the case of foundation and rouge or eyeshadow, or of interaction with the saliva in the case of lipsticks. Moreover, this alteration in colour is frequently not uniform, requiring the user to apply fresh makeup at frequent intervals, which may constitute a loss of time.

The problems of colour stability were hitherto solved by introducing a high proportion of pigments into the compositions, such that said compositions deposit a large amount of pigments on the substrate to which they are applied.

Compositions have also been proposed which include volatile compounds which, although exhibiting enhanced properties of staying power, may have the drawback, following evaporation of the volatile compounds, of leaving on the skin and lips a film which gradually becomes uncomfortable (a sensation of dryness and tautening), which has led a certain number of women to shun this type of lipstick.

Furthermore, these compositions may lead to matte, opaque coloured films. However, women today are on the look-out for products, such as for colouring the lips or eyelids, which are glossy and semi-opaque.

It is known, moreover, that enhancing the gloss properties requires effective dispersion of the solid particles in the composition, such as of the pigments.

U.S. Pat. No. 5,945,092 assigned to Revlon (“the '092 patent”) thus describes the use of silicone surfactants in combination with volatile oils and fluorinated dispersants. However, as explained above, the use of volatile compounds involves certain disadvantages. Moreover, despite their effectiveness, these surfactants have the drawback of being potentially irritant, such as for the labial mucosa, when present in a high percentage in the composition (typically greater than 3%), and all the more so when the proportion of volatile oil is high (typically greater than 30%). The fluorinated dispersants described in the examples of the '092 patent do not have the solubility parameters, which are within the ranges of those as defined herein.

The company Kao, in its application EP-A-0548694, proposed a composition comprising a silicone surfactant (polyether-modified silicone), oils and pigments, possessing good use comfort and enhanced staying power. Nevertheless, these compositions do not allow a makeup possessing sufficient staying power to be obtained.

Moreover, at present in the field of cosmetology, preference is given to the use of compounds natural in origin. The silicone and fluorinated surfactants provided in the compositions of the above-mentioned documents, however, are synthetic in origin.

A European Application EP 1 112 734 describes compositions comprising a hydrocarbon surfactant, a non-volatile silicone compound and a fat-soluble polymer (in the form of a paste). However, the hydrocarbon surfactants given as examples (such as diglyceryl triisostearate or poly(12-hydroxystearic) acid) have a solubility parameter δₐ of between 2 and 9.08 (J/cm³)^{1/2}. Comparative Example 2 of the present application shows that a composition containing a dispersant such that the parameter δₐ is between 9.1 and 20 (J/cm³)^{1/2} allows the staying power of the makeup to be enhanced while retaining the same gloss.

There is therefore still a need for a composition which does not have at least one of the above drawbacks and which can possess, for example, enhanced staying power properties and little or no migration while giving the applied makeup or care product a more or less glossy appearance, appropriate to the wishes of the consumer, which does not dry out and does not tauten the skin or the lips to which it is applied, either during application or over time, and which is not irritant to the skin or lips.

The inventors have found surprisingly that the use of a combination of at least one non-volatile silicone compound, at least one non-silicone fat-soluble polymer and at least one hydrocarbon dispersant allows a composition to be obtained which can have enhanced staying power, such as with regard to colour, which can be glossy, comfortable and exhibits little or no migration, and which is not irritant.

Disclosed herein is an anhydrous care or makeup composition for a keratin material comprising, in a cosmetically acceptable medium, at least one non-volatile silicone compound, at least one non-silicone fat-soluble polymer and at least one hydrocarbon dispersant which has solubility parameters δd and δa meeting the following conditions: 16.2≦δ_{d}≦20 (J/cm³)^{1/2} and 9.1≦δₐ≦20 (J/cm³)^{1/2}, wherein the at least one hydrocarbon dispersant comprising carbon and hydrogen atoms and at least one functional group chosen from hydroxyl, ester, ether, carboxylic acid and amide functional groups.

The term “at least” one compound means one or more compounds.

The term “anhydrous cosmetic composition” means a composition comprising a total content of water of no more than 5% by weight of the composition, such as no more than 2% by weight, and further such as no more than 0.5% by weight of the composition.

Further disclosed herein is a cosmetic method of imparting to a film of anhydrous cosmetic composition at least one property chosen from properties of colour fastness, gloss, comfort and non-migration, comprising introducing into said composition at least one non-volatile silicone compound, at least one non-silicone fat-soluble polymer and at least one hydrocarbon dispersant which has solubility parameters δ_{d} and δₐ meeting the following conditions: 16.2≦δ_{d}≦20 (J/cm³)^{1/2} and 9.1≦δₐ≦20 (J/cm³)^{1/2}.

Even further disclosed herein is the use of a combination of at least one non-volatile silicone compound, at least one non-silicone fat-soluble polymer and at least one hydrocarbon dispersant which has solubility parameters δ_{d} and δₐ meeting the following conditions: 16.2≦δ_{d}≦20 (J/cm³)^{1/2} and 9.1≦δₐ≦20 (J/cm³)^{1/2}, in an anhydrous cosmetic composition which can have enhanced staying power, such as good colour fastness, and can have at least one property chosen from properties of being glossy, comfortable and non-migrating.

Even further disclosed herein is the use of a combination of at least one non-volatile silicone compound, at least one non-silicone fat-soluble polymer and at least one hydrocarbon dispersant which has solubility parameters δ_{d} and δₐ meeting the following conditions: 16.2≦δ_{d}≦20 (J/cm³)^{1/2} and 9.1≦δₐ≦20 (J/cm³)^{1/2}, in a cosmetic composition as an agent for imparting to said composition at least one property chosen from properties of staying power, such as colour fastness, gloss, comfort and non-migration.

The at least one non-volatile silicone compound, the at least one non-silicone fat-soluble polymer and the at least one hydrocarbon dispersant of the composition disclosed herein are distinct compounds.

The term “keratin material” means skin, lips, hair, and exoskeletal appendages.

A “non-volatile” compound means a compound capable of remaining on the skin or lips for several hours. A non-volatile compound has, for example, at room temperature and atmospheric pressure, a non-zero vapour pressure less than 0.02 mm Hg (2.66 Pa).

A “volatile” compound means a compound capable of evaporating from the skin or lips in less than one hour. A volatile compound is chosen, for example, from compounds having a vapour pressure, at room temperature and atmospheric pressure, ranging from 0.02 mm to 300 mm Hg (2.66 Pa to 40 000 Pa) and such as ranging from 0.1 to 90 mm Hg (13 Pa to 12 000 Pa).

A “fat-soluble polymer” means a polymer which is non-waxy, liquid or pasty at room temperature (25° C.) and which is soluble in the oils as disclosed herein.

The composition disclosed herein comprises ingredients which can be compatible with the keratin materials, such as the skin, lips, keratin fibres and nails. It may be provided in a fluid form, in the form of a paste or rigid or non-deformable solid, cast where appropriate in the form of a stick or dish. It is, for example, provided in fluid form or stick form, such as an anhydrous form of this kind. The term “fluid” means a composition which flows under its own weight, in contrast to a solid.

The composition comprises few or no volatile oils and such as less than 10% relative to the total weight of the composition, further such as less than 5% and even further such as less than 2% relative to the total weight of the composition, and in one embodiment, the composition is free from volatile oil.

The at least one hydrocarbon dispersant used in the composition can serve to prevent the dispersed particles from agglomerating or flocculating. A “hydrocarbon” compound means a compound comprising carbon and hydrogen atoms and at least one functional group chosen from hydroxyl, ester, ether (ethylene oxide), carboxylic acid and amide functional groups.

The at least one hydrocarbon dispersant has no fluorine atoms. This dispersant carries at least one functionality having a strong affinity for the surface of the particles to be dispersed.

The at least one hydrocarbon dispersant of the composition is, for example, fluid at room temperature (25° C.). In another embodiment, the at least one hydrocarbon dispersant of the composition possesses at least one of the following characteristics: being liquid and having a refractive index ≧1.45 at 20° C., wherein the refractive index is measured on a refractometer.

This at least one hydrocarbon dispersant has solubility parameters δ_{d} and δₐ in accordance with the Hansen solubility space which meet the following conditions:

16.2≦_{d}≦20 (J/cm³)^{1/2}, such as 16.3≦δ_{d}≦19 (J/cm³)^{1/2}, and further such as 16.9≦δ_{d}≦18 (J/cm³)^{1/2} and

9.1≦δₐ≦20 (J/cm³)^{1/2}, such as 10≦δₐ≦18.1 (J/cm³)^{1/2}, and further such as 13≦δₐ≦14.5 (J/cm³)^{1/2}.

The definition of the solubility parameters according to Hansen is well known to the person skilled in the art and is described, for example, in the article by C. M. Hansen: “The three dimensional solubility parameters” J. Paint Technol. 39, 105 (1967). These parameters are also described in the document JP-A-08-109121 from Kao and the document by D. W. Van Krevelen: “Properties of polymers” (1990), p. 190.

According to the Hansen solubility space:

δ_{d} characterizes the London dispersion forces resulting for the formation of dipoles induced during molecular impacts;

δₚ characterizes the Debye interaction forces between permanent dipoles;

δₕ characterizes the specific interaction forces (of hydrogen-bond, acid/base, donor/acceptor type, etc.); and

δₐ=(δₚ²+δₕ²)^{1/2}.

The parameters δ_{d}, δₚ and δₕ are generally expressed in (J/cm³)^{1/2}. They are determined at room temperature (25° C.) and, for example, according to the calculation method indicated in the above patent document from Kao.

In the composition disclosed herein it is possible to use any fluid, and such as liquid, hydrocarbon dispersant or mixture of fluid hydrocarbon dispersants which meets the above conditions. Where a mixture of fluid hydrocarbon dispersants is used, the solubility parameters of the mixture are determined from those of the fluid hydrocarbon dispersants taken separately, according to the following relationships:

$${δ}_{D mix} = {\sum }_{i} x i {δ}_{D i} ; {δ}_{p mix} = {\sum }_{i} x i {δ}_{D i} ; {δ}_{p i} and {δ}_{h m i x} = {\sum }_{i} x i {δ}_{h i}$$

wherein xi represents the volume fraction of the fluid hydrocarbon dispersant (i) in the mixture.

It is within the scope of the person skilled in the art to determine the amounts of each fluid hydrocarbon dispersant so as to obtain a mixture of fluid hydrocarbon dispersants which meets the above conditions.

In one embodiment, the at least one hydrocarbon dispersant has a chemical structure which comprises at least one polar group chosen from —COOH; —OH; ethylene oxide —(O—CH₂—CH₂—); propylene oxide

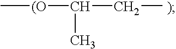

—PO₄; NHR; NR₁R₂ wherein R₁ and R₂, which may be identical or different, optionally form a ring and are each chosen from linear and branched C₁ to C₂₀ alkyl and alkoxy radicals, and

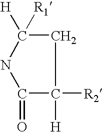

wherein R₁′ and R₂′, which may be identical or different, are each chosen from H and linear and branched C₁ to C₂₀ alkyl and alkoxy radicals.

The at least one hydrocarbon dispersant may be chosen from:

ether-modified fatty alcohols and such as the addition products of at least one compound chosen from ethylene oxide and propylene oxide with at least one other compound chosen from linear and branched fatty alcohols and alkylphenols,

esters resulting from the reaction of at least one fatty acid with at least one addition product chosen from addition products of ethylene oxide and glycerol and addition products of ethylene oxide and polyglycerol,

esters resulting from the reaction of at least one compound chosen from glycerol and polyglycerol with at least one addition product of ethylene oxide and a fatty acid chosen from saturated and unsaturated fatty acids,

partial esters resulting from the reaction of at least one compound chosen from saturated and unsaturated, linear and branched fatty acids, ricinoleic acid and 12-hydroxystearic acid with at least one polyol such as glycerol, polyglycerol, pentaerythritol, saccharide alcohols such as sorbitol, and such as esters of polyglycerol,

esters resulting from the reaction of sorbitan with at least one fatty acid chosen from saturated and unsaturated, linear and branched fatty acids,

ether-modified sorbitan esters, such as esters resulting from at least one reaction chosen from the reaction of sorbitan with at least one addition product of ethylene oxide and a fatty acid chosen from saturated and unsaturated fatty acids and the reaction of at least one fatty acid chosen from saturated and unsaturated fatty acids with at least one addition product of ethylene oxide and sorbitan,

addition products of ethylene oxide with at least one compound chosen from castor oil and hydrogenated castor oil,

trialkyl phosphates and alkyl mono-, di- and triphosphates,

these compounds satisfying the solubility parameters defined above.

The term “ester” means a monoester, a diester, a triester and, more generally, a polyester.

The at least one hydrocarbon dispersant is, for example, chosen from monoesters, diesters and esters resulting from partial esterification, i.e., the final ester comprises at least one free —OH functional group.

The at least one hydrocarbon dispersant can further, for example, be chosen from:

addition products of at least one compound chosen from ranging from 2 to 30 moles of ethylene oxide and ranging from 0 to 5 moles of propylene oxide with at least one other compound chosen from linear and branched C₈ to C₄₀, such as C₈ to C₂₂, fatty alcohols and alkylphenols,

esters resulting from the reaction of at least one fatty acid chosen from C₈ to C₄₀, such as C₈ to C₂₂, fatty acids with at least one addition product chosen from addition products of from 1 to 30 moles of ethylene oxide and glycerol and addition products of from 1 to 30 moles of ethylene oxide and polyglycerol,

esters resulting from the reaction of at least one compound chosen from glycerol and polyglycerol with at least one addition product of from 2 to 30 moles of ethylene oxide and a fatty acid chosen from C₈ to C₄₀, such as C₈ to C₂₂, saturated and unsaturated fatty acids,

partial esters resulting from the reaction of at least one compound chosen from saturated and unsaturated C₈ to C₄₀, such as C₈ to C₂₂, linear and branched fatty acids, ricinoleic acid, and 12-hydroxystearic acid with at least one other compound chosen from glycerol, polyglycerol, pentaerythritol and sorbitol,

esters resulting from the reaction of sorbitan with at least one fatty acid chosen from saturated and unsaturated C₈ to C₄₀, such as C₈ to C₂₂, linear and branched fatty acids,

esters resulting from at least one reaction chosen from the reaction of sorbitan with at least one addition product of from 2 to 30 moles of ethylene oxide and a fatty acid chosen from saturated and unsaturated C₈ to C₄₀, such as C₈ to C₂₂, fatty acids and the reaction of at least one fatty acid chosen from C₈ to C₄₀, such as C₈ to C₂₂, saturated and unsaturated fatty acids with at least one addition product of from 2 to 30 moles of ethylene oxide and sorbitan,

addition products of from 2 to 60 moles of ethylene oxide with at least one compound chosen from castor oil and hydrogenated castor oil,

trialkyl phosphates and alkyl mono-, di- and triphosphates.

The at least one hydrocarbon dispersant is, even further, for example, chosen from:

ethoxylated myristyl alcohol comprising 15 ethylene oxide groups (or EO) (δ_{d}=17.33 (J/cm³)^{1/2} and δₐ=9.28 (J/cm³)^{1/2}), ethoxylated polyglyceryl-2 monoisostearate comprising 5 EO (δ_{d}=17.34 (J/cm³)^{1/2} and δₐ=12.22 (J/cm³)^{1/2}), polyglyceryl-3 diisostearate (δ_{d}=16.96 (J/cm³)^{1/2} and δₐ=10.4 (J/cm³)^{1/2}), glyceryl monoiso-stearate (δ_{d}=16.32 (J/cm³)^{1/2} and δₐ=11.01 (J/cm³)^{1/2}), polyglyceryl-2 monoisostearate (δ_{d}=17.03 (J/cm³)^{1/2} and δₐ=13.25 (J/cm³)^{1/2}), polyglyceryl-3 isostearate (δ_{d}=17.38 (J/cm³)^{1/2} and δₐ=14.48 (J/cm³)^{1/2}), polyglyceryl-4 isostearate (δ_{d}=17.57 (J/cm³)^{1/2} and δₐ=15.37 (J/cm³)^{1/2}), polyglyceryl-6 monoisostearate (δ_{d}=17.86 (J/cm³)^{1/2} and δₐ=16.61 (J/cm³)^{1/2}), polyglyceryl-10 monoisostearate (δ_{d}=18.22 (J/cm³)^{1/2} and δₐ=18.41 (J/cm³)^{1/2}), polyglyceryl-2 monooleate (δ_{d}=17.14 (J/cm³)^{1/2} and δₐ=13.39 (J/cm³)^{1/2}), sorbitan isostearate (δ_{d}=17.33 (J/cm³)^{1/2} and δₐ=13.56 (J/cm³)^{1/2}), sorbitan monooleate (δ_{d}=17.32 (J/cm³)^{1/2} and δₐ=13.66 (J/cm³)^{1/2}), and ethoxylated sorbitan monooleate comprising 5 EO (δ_{d}=17.56 (J/cm³)^{1/2} and δₐ=12.47 (J/cm³)^{1/2}).

In one embodiment, the at least one hydrocarbon dispersant is chosen from partial esters of polyglycerol and isostearic acid, partial esters of polyglycerol and oleic acid, and partial esters of sorbitan and oleic acid.

In another embodiment, the at least one hydrocarbon dispersant is chosen from polyglyceryl-2 monoisostearate such as Salacos 41, manufactured or sold by Nisshin Oil Mills, polyglyceryl-3 diisostearate such as Lameform TGI, manufactured or sold by Cognis, polyglyceryl-2 monooleate such as Rylo PG 29, manufactured or sold by Danisco Ingredients, and sorbitan monooleate such as Span 80, manufactured or sold by Uniqema.

The amounts of the various ingredients in the composition will be given as percentages by weight relative to the total weight of the composition.

The at least one hydrocarbon dispersant may represent, for example, from 0.5 to 40%, such as from 3 to 20% and further such as from 5 to 15% of the total weight of the composition.

The at least one non-volatile silicone compound used in the composition is, for example, a non-aqueous compound and such as a cosmetic oil. The term “oil” means any non-aqueous liquid medium which is insoluble in water at room temperature (25° C.) and atmospheric pressure (760 mm Hg or 1.01×10⁵ Pa).

For example, the at least one non-volatile silicone compound has a viscosity which is in the range, for example, from 5 to 1 000 000 cSt at 25° C., such as from 10 to 500 000 cSt at 25° C., and further such as 10 to 5 000 cSt at 25° C. The viscosity is measured according to ASTM D-445.

By way of examples of the at least one non-volatile silicone compound which may be used in the composition disclosed herein, mention may be made of polydimethylsiloxanes, (PDMS), phenylated silicones, polyalkylmethylsiloxanes, silicone resins such as those described in the documents JP-A-62-61911, JP-A-61-65809 and EP-A-602905, fluorinated silicones and mixtures thereof.

The at least one non-volatile silicone compound is chosen, for example, from non-volatile polydimethylsiloxanes; polydimethylsiloxanes comprising at least one group chosen from alkyl, alkoxy and phenyl groups, pendently or at the silicone chain end, wherein the at least one group comprises from 2 to 24 carbon atoms; phenyltrimethcones, phenyldimethicones, phenyltrimethylsiloxydiphenylsiloxanes, diphenyldimethicones, diphenylmethyldiphenyltrisiloxanes and 2-phenylethyl trimethylsiloxysilicates; fluorinated silicones comprising at least one fluorinated group pendently or at the silicone chain end, comprising from 1 to 12 carbon atoms, some or all of the hydrogens thereof being substituted by fluorine atoms; silicone resins; and silicone gums, including dimethiconols.

The at least one non-volatile silicone compound may represent, for example, from 0.5 to 90%, such as from 5 to 60% and further such as from 10 to 50% of the total weight of the composition.

The composition disclosed herein further comprises at least one non-silicone fat-soluble polymer.

The at least one non-silicone fat-soluble polymer has, for example, a molecular mass ranging from 400 to 500 000 g/mol and such as from 500 to 100 000 g/mol.

The at least one non-silicone fat-soluble polymer is chosen from vinyl polymers, fat-soluble polyethers, and non-crosslinked polyesters.

The at least one non-silicone fat-soluble polymer may be chosen, for example, from:

a) vinyl polymers, such as:

branched non-crystalline olefin homopolymers,

non-crystalline non-branched olefin copolymers,

non-crystalline hydrogenated diene homopolymers and copolymers,

linear and branched oligomers, alkyl (meth)acrylate homopolymers and copolymers such as those having a C₈-C₃₀ alkyl group and a molar mass of less than or equal to 10 000 g/mol, perfluorinated alkyl (meth)acrylate oligomers, homopolymers and copolymers such as those having a C₈-C₃₀ alkyl group and a molar mass of less than or equal to 10 000 g/mol, for example, less than or equal to 8 000 g/mol,

vinyl ester oligomers, homopolymers and copolymers having at least one alkyl group chosen from C₈-C₃₀ alkyl groups and a molar mass of less than or equal to 10 000 g/mol, such as less than or equal to 8 000 g/mol,

vinyl ether oligomers, homopolymers and copolymers having at least one alkyl group chosen from C₈-C₃₀ alkyl groups and a molar mass of less than or equal to 10 000 g/mol, such as less than or equal to 8 000 g/mol,

b) fat-soluble polyethers resulting from the polyetherification of at least one diol chosen from C₂-C₁₀₀ diols, such as C₂-C₅₀ diols, and

c) non-crosslinked polyesters resulting from the polycondensation of an acid chosen from C₄-C₅₀ linear and branched dicarboxylic and polycarboxylic acids and a compound chosen from C₂-C₅₀ diols and polyols.

The at least one non-silicone fat soluble polymer in the composition may be, for example chosen from:

olefin homopolymers, such as:

polybutylenes such as Indopol H-100 (of molar mass or MM=965 g/mol), Indopol H-300 (MM=1 340 g/mol) and Indopol H-1500 (MM=2 160 g/mol), produced or sold by Amoco,

hydrogenated polyisobutylenes such as Panalane H-300E (MM=1 340 g/mol), produced or sold by Amoco, Viseal 20000 (MM=6 000 g/mol), produced or sold by Synteal, Rewopal PIB 1000 (MM=1 000 g/mol), produced or sold by Witco,

polydecenes and hydrogenated polydecenes such as Puresyn 8 (MM=635 g/mol), Puresyn 10 (MM=723 g/mol) and Puresyn 150 (MM=9 200 g/mol), produced or sold by Mobil Chemicals;

non-branched olefin copolymers, such as:

vinylpyrrolidone (VP)/olefin copolymers having a number of carbons ranging from 8 to 30, for example, from 10 to 30, such as the vinylpyrrolidone/1-hexadecene copolymer produced or sold by ISP under the name Antaron V-216 (MM=7 300 g/mol);

non-crystalline hydrogenated diene homopolymers and copolymers, such as:

hydrogenated polybutadiene, and hydrogenated polyisoprene;

vinyl ester oligomers, homopolymers and copolymers, such as:

polyvinyl laurate (homopolymer of ethenyl dodecanoate), such as Mexomère PP produced or sold by Chimex,

vinyl acetate/allyl stearate copolymer such as Mexomère PQ produced or sold by Chimex; and

fat-soluble polyethers, such as:

PEG-45/dodecyl glycol copolymer (block polymer of polyoxyethylene/polydodecyl glycol) such as Elfacos ST 9, produced or sold by Akzo Nobel.

The at least one non-silicone fat-soluble polymer may represent, for example, from 1 to 98.5%, such as from 2 to 85%, further such as from 5 to 70%, and even further such as from 5 to 60% of the total weight of the composition.

The composition disclosed herein may, for example, further comprise at least one colorant, which may be chosen from dyes which are soluble or dispersible in the composition, pigments, and nacres and nacreous pigments. The dyes are, for example, fat-soluble dyes, although water-soluble dyes may be used. The at least one colorant may represent, for example, from 0.001 to 98%, such as from 0.5 to 85%, and further such as from 1 to 60% of the total weight of the composition.

For a composition in paste or cast form such as lipsticks, foundations or body makeup products, the at least one colorant represents generally, for example, from 0.001 to 60%, such as from 2 to 40% and further such as from 5 to 30%, relative to the total weight of the composition.

The fat-soluble dyes are, for example, Sudan red, D & C Red 17, D & C Green 6, β-carotene, soya oil, Sudan brown, D & C Yellow 11, D & C Violet 2, D & C Orange 5, quinoline yellow and annatto. They may represent, for example, from 0 to 20% of the weight of the composition and such as from 0.1 to 6% of the total weight of the composition. The water-soluble dyes are, for example, beetroot juice and methylene blue, and may represent, for example, from 0.1 to 6% of the weight of the composition (if present).

The composition disclosed herein comprises, for example, a particulate phase which may represent, for example, from 0.001 to 50% of the total weight of the composition, such as from 0.01 to 40%, further such as from 0.5 to 25%, and even further such as from 0.05 to 30% of the total weight of the composition, and which may comprise at least one component chosen from pigments, nacres, and fillers which are commonly used in cosmetic compositions.

The term “pigments” means particles chosen from organic and inorganic, white and coloured particles which are insoluble in the liquid fatty phase and are intended for colouring and/or opacifying the composition. The term “fillers” means particles chosen from mineral and synthetic, white and colourless particles which are lamellar or non-lamellar. The term “nacres” means iridescent particles, produced, for example, by certain molluscs in their shell or synthesized. These fillers and nacres may serve, for example, to modify the texture of the composition.

The pigments may be present in the composition in a proportion of, for example, from 0.05 to 30% (if present) of the total weight of the composition, and such as from 2 to 20% of the total weight of the composition. The inorganic pigments which can be used are, for example, titanium oxide, zirconium oxide and cerium oxide and also the oxides of zinc, iron and chromium, and iron blue. The organic pigments which can be used are, for example, carbon black and the lakes of barium, strontium, calcium (D & C Red No. 7) and aluminium.

The nacres may be present in the composition in a proportion of, for example, from 0.001 to 20% (if present) of the total weight of the composition, such as from 1 to 15% of the total weight of the composition. The nacres which can be used are, for example, mica covered with titanium oxide, with iron oxide, with natural pigment or with bismuth oxychloride, such as coloured titanium mica, goniochromatic pigments and, for example, multilayer interference pigments.

The fillers may be presentin a proportion of, for example, from 0.001 to 35% (if present) of the total weight of the composition, such as from 0.5 to 15% of the total weight of the composition. Fillers that may be used are, for example, talc, mica, kaolin, lauroyl lysine, polyamide powders such as Nylon® (Orgasol, for example) and polyethylene powders, polytetrafluoroethylene powders (Teflon®), starch, boron nitride, copolymer microspheres such as Expancel® (Nobel Industrie), Polytrap® (Dow Corning), Polypore® L 200 (Chemdal Corporation) and silicone resin microbeads (Tospearl® from Toshiba, for example).

The composition disclosed herein may further comprise at least one additional non-aqueous compound other than the at least one non-volatile silicone compound, the at least one non-silicone fat-soluble polymer and the at least one hydrocarbon dispersant, chosen from oils, fatty substances which are pastelike at room temperature, waxes, gums, and resins.

In one embodiment, the composition further comprises at least one wax. The term “wax” means a lipophilic fatty compound which is solid at room temperature (25° C.) with a reversible solid/liquid state change, having a melting temperature of more than 30° C., such as greater than 45° C., possibly ranging up to 200° C., a hardness of more than 0.5 MPa, and anisotropic crystalline organization in the solid state. The size of the crystals is such that the crystals diffract and/or diffuse light, giving the composition a more or less opaque, cloudy appearance. By taking the wax to its melting temperature it is possible to make it miscible with the oils and to form a microscopically homogeneous mixture, but by taking the temperature of the mixture back to room temperature the wax is recrystallized in the oils of the mixture.

The at least one wax which can be used is chosen, for example, from those which are generally used in the cosmetics field: for example, waxes of natural origin such as beeswax, carnauba wax, candelilla wax, ouricury wax, Japan wax, cork fibre wax and sugarcane wax, rice wax, montan wax, paraffin, lignite wax and microcrystalline wax, ceresin and ozokerite, hydrogenated oils such as jojoba oil and hydrogenated castor oil; synthetic waxes such as polyethylene waxes obtained from the polymerization or copolymerization of ethylene, and Fischer-Tropsch waxes and esters of fatty acids such as octacosanyl stearate, glycerides which are solid at 30° C., glycerides which are solid at 45° C., silicone waxes such as alkyl- and alkoxy-dimethicones comprising at least one radical chosen from alkyl and alkoxy radicals of from 10 to 45 carbon atoms, poly(di)methylsiloxane esters which are solid at 30° C. and whose ester chain comprises at least 10 carbon atoms, and di(1,1,1-trimethylol-propane) tetrastearate, manufactured or sold by the company Heterene under the name HEST 2T-4S; and mixtures thereof.

The nature and the amount of the gums, pastelike substances or waxes are a function of the desired mechanical and textural properties. For example, the composition may comprise from 0.01 to 50% by weight of waxes, relative to the total weight of the composition, such as from 2 to 40% and further such as from 5 to 30% by weight of waxes, relative to the total weight of the composition.

The additional oils may be chosen from hydrocarbon and fluorinated oils. These oils may be of animal, plant, mineral or synthetic origin. The term “hydrocarbon oil” means an oil comprising mainly carbon and hydrogen atoms and optionally at least one functional group chosen from hydroxyl, ester, ether and carboxy functional groups.

The oils which can be used in the composition are, for example:

hydrocarbon oils of animal origin such as perhydrosqualene;

hydrocarbon oils of vegetable origin such as the liquid triglycerides of fatty acids comprising from 4 to 24 carbon atoms, such as the triglycerides of heptanoic or octanoic acids, and sunflower oil, maize oil, soya oil, marrow oil, grape seed oil, sesame oil, hazelnut oil, apricot oil, macadamia oil, castor oil, avocado oil, capric/caprylic acid triglycerides such as those sold by Stéarineries Dubois and those sold under the names Miglyol 810, 812 and 818 by Dynamit Nobel, jojoba oil, karite butter, squalane of synthetic or vegetable origin;

linear or branched hydrocarbons of mineral or synthetic origin such as liquid paraffins and derivatives thereof, such as vaseline;

synthetic ethers and esters, such as of fatty acids, such as the oils of formula R₁COOR₂ in which R₁ is the residue of a higher fatty acid comprising from 1 to 40 carbon atoms and R₂ is a hydrocarbon chain comprising from 1 to 40 carbon atoms, with R₁+R₂≧10, for example, purcellin oil, isononyl isononanoate, isopropyl myristate, 2-ethylhexyl palmitate, 2-octyldodecyl stearate, 2-octyldodecyl erucate, isostearyl isostearate; hydroxylated esters such as isostearyl lactate, octyl hydroxystearate, octyidodecyl hydroxystearate, diisostearyl malate, triisocetyl citrate, heptanoates, octanoates and decanoates of fatty alcohols; polyolesters such as propylene glycol dioctanoate, neopentyl glycol diheptanoate, diethylene glycol diisononanoate; and pentaerythritol esters such as pentaerythrityl tetraisostearate;

fatty alcohols comprising from 12 to 26 carbon atoms such as octyldodecanol, 2-butyloctanol, 2-hexyl-decanol, 2-undecylpentadecanol, and oleyl alcohol;

fluorinated oils which have optionally been partially hydrocarbon-treated, such as methoxy-nonafluorobutane; and

mixtures thereof.

The additional oils of the composition may make up, for example, from 0.1% to 90% of the total weight of the composition, such as from 5 to 60% and further such as from 10 to 50% of the total weight of the composition.

The composition disclosed herein may further comprise at least one additive commonly used in the field in question, such as antioxidants, preservatives, neutralizing agents, lipophilic gelling agents and liquid non-aqueous compounds, dispersants, active cosmetic substances, and mixtures thereof. The at least one additive may be present in the composition in a proportion of, for example, from 0.0005 to 20% of the total weight of the composition and such as from 0.001 to 10% of the total weight of the composition.

The term “active cosmetic substance” means a compound chosen from lipophilic and hydrophilic compounds imparting a benefit to a keratin material, such as the skin and lips.

The active cosmetic substances which can be used are, for example, vitamin A, vitamin E, vitamin C, vitamin B₃, vitamin F, provitamins such as D-panthenol, active soothing agents such as α-bisabolol, aloe vera, allantoin, plant extracts and essential oils, protective or restructuring agents such as ceramides, active freshness agents such as menthol and its derivatives, emollients (cocoa butter, dimethicone), moisturizers (arginine PCA), active anti-wrinkle agents, essential fatty acids, sunscreens, and mixtures thereof.

The person skilled in the art will of course take care to select the nature and/or quantity of any complementary additives such that the advantageous properties of the composition disclosed herein are not, or not substantially, adversely affected by the addition envisaged.

In one embodiment, the composition may be prepared conventionally by the person skilled in the art. The composition may be provided in the form of a cast product and, for example, in the form of a stick or tube, or in compacted form, for example in the form of a dish which can be used by direct contact or by sponge, or in a bottle. For example, the composition can be used as cast foundation, blusher or eyeshadow, lipstick, lipcare base or balm, or concealer. The composition may also be provided in the form of a flexible paste or a gel or a fluid cream, constituting foundations or lipsticks, lip glosses, sun products or skin colouring products.

In such cases the composition may be present, for example, in the form of an oily gel, oily liquid, paste or stick or in the form of a vesicular dispersion comprising at least one lipid chosen from ionic and non-ionic lipids. These preparation forms are prepared in accordance with the customary methods of the fields in question.

The composition may also be provided in the form of a coloured or colourless skin care composition, in the form of a sun protection or makeup remover composition or in the form of a hygiene composition. If the composition contains active cosmetic substances, it may then be used as a care base or non-therapeutic treatment base for skin, such as the hands or face, or for the lips, such as lip balms, protecting lips from at least one external factor chosen from the cold, the sun and the wind.

The composition may also be provided in the form of a coloured makeup product for the skin, for example, a face makeup product such as a foundation, a blusher, a rouge or eyeshadow, a body makeup product such as a semi-permanent tattooing product or a lip makeup product such as a lipstick or lip gloss, possibly having care or non-therapeutic treatment properties, a product for making up the exoskeletal appendages, for example, a nail varnish, mascara or eyeliner.

In one embodiment, the composition is provided in the form of lipstick or lip gloss.

The composition should be physiologically acceptable (cosmetically acceptable), i.e. non-toxic and capable of being applied to the skin, exoskeletal appendages or lips of human beings.

The term “cosmetically acceptable” means pleasant in at least one aspect chosen from aspects of taste, feel, appearance and odour.

The invention is illustrated in greater detail in the following examples. The percentages are by mass.

### EXAMPLES 1 AND 2

### Lipsticks

The compositions which appear in the table (I) below were produced.

The composition of Example 1 (according to the present invention) comprises, as inventive dispersant, sorbitan monooleate (δd=17.32 (J/cm³)^{1/2} and δa=13.66 (J/cm³)^{1/2}), manufactured or sold by Uniqema under the reference Span 80 V.

In the composition of Example 2 (comparative), the 13.4% of sorbitan monooleate were replaced by 10% of polyglyceryl-2 triisostearate, manufactured or sold by Nisshin Oil Mills under the reference Salacos 43 (δd=16.7 (J/cm³)^{1/2} and δa=6.69 (J/cm³)^{1/2}).

TABLE (I)

| | | Example 1 | Example 2 |
|---|---|---|---|
| Phase | | (inventive) | (comparative) |
| A- | Tridecyl trimellitate | 4.4 | 4.4 |
| | Vinylpyrrolidone/1-hexa-decene | 1.6 | 1.6 |
| | copolymer sold or manufactured | | |
| | by ISP under the reference | | |
| | Antaron V-216 | | |
| | Vinylpyrrolidone/1-eicosene | 0.8 | 0.8 |
| | copolymer sold or manu-factured | | |
| | by ISP under the reference | | |
| | Antaron V-220 | | |
| | Bisdiglyceryl polyacyl-adipate-2 | 1.6 | 1.6 |
| | BHT | 0.04 | 0.04 |
| | Polyglyceryl-2 triiso-stearate | — | 13.4 |
| | Sorbitan monooleate | 13.4 | — |
| | Isononyl isononanoate | 10 | 10 |
| B- | Polyethylene wax (MM = | 6.6 | 6.6 |
| | 500 g/mol) | | |
| | Octacosanyl stearate | 5.5 | 5.5 |
| C- | Red 21 | 0.06 | 0.06 |
| | Red 7 | 0.2 | 0.2 |
| | Iron oxides (Cl 77491 and | 2.2 | 2.2 |
| | Cl 77499) | | |
| C′- | Ethylene glycol methacrylate/ | 1 | 1 |
| | lauryl methacrylate copolymer | | |
| | manufactured or sold under the | | |
| | reference Polytrap 603 by | | |
| | Advanced Polymer Systems | | |
| | N-Lauroyl-L-lysine | 2.5 | 2.5 |
| | Kaolin | 5 | 5 |
| D- | Hydrogenated isoparaffin | qs 100 | qs 100 |
| | manufactured or sold under the | | |
| | reference Parléam by Nippon Oil | | |
| | and Fats | | |
| | Hydrogenated polyisobutene | 8.5 | 8.5 |
| | manufactured or sold under the | | |
| | reference Panalane H-300E by | | |
| | Amoco Chemical | | |
| | Phenyltrimethicone sold or | 12.7 | 12.7 |
| | manufactured by Dow Corning | | |
| | under the reference DC 556 | | |
| | Pyrogenic silica | 3 | 3 |
| E- | Titanium oxide mica | 1.8 | 1.8 |

Procedure:

The pigments (phase C) and fillers (phase C′) were ground in phase A. In parallel, a silica gel was prepared (phase D) by mixing the silica into the hydrogenated isoparaffin, the phenyltrimethicone and the hydrogenated poly-isobutene.

Then the ground product (phases A+C+C′) and the silica gel (phase D) and the waxes (phase B) were added in a heating vessel and heated at 100° C. for 2 hours, and homogenized.

Finally, the nacre (phase E) was added to the mixture, which was cast in an appropriate mould at 42° C. The mould was subsequently placed at −20° C. for half an hour, after which the sticks were demoulded.

Cosmetic Evaluation:

The staying power of the two formulas was evaluated with the aid of instrumental and sensorial methods on a panel of 12 experienced individuals who applied each of the formulas one after the other.

The staying power was evaluated as follows:

Firstly, the staying power was evaluated one hour after application of the formula to the lips.

Secondly, the staying power was evaluated after a series of tests which consist in making two “kiss marks” on a paper tissue, drinking a hot drink and then a cold drink and eating 4 mouthfuls of a sandwich and an apple.

The instrumental staying power was evaluated on a scale ranging from 1 to 100:1 corresponds to a formula which does not hold at all and 100 to a formula which holds very well. The difference between two results is significant if it is greater than or equal to 10.

The migration, gloss and comfort were also evaluated by the 12 individuals:

the gloss was evaluated immediately after application of the formula and then after one hour

the migration and comfort were evaluated after one hour.

The composition of Example 1 possesses better staying power properties than the composition of Example 2 (the staying power was evaluated at 71 for the composition of Example 1 against a value of 59 for the composition of Example 2), while being equivalent in gloss, comfort and migration. Moreover, the gloss of the film of the composition of Example 1 lasts longer.

### EXAMPLES 3 AND 4

### Lipsticks

The properties of two compositions according to the present invention and the prior art were compared. The composition of Example 3 according to the present invention comprises a hydrocarbon dispersant, namely polyglyceryl-2 monoisostearate (manufactured or sold by Nisshin Oils Mills under the reference Salacos 41) and the composition of Example 4 (comparative) comprises a silicone dispersant, namely cetyl dimethicone copolyol (manufactured or sold by Goldschmidt under the reference Abil EM 90 Desodorise).

TABLE (II)

| | | Example 3 | Example 4 |
|---|---|---|---|
| Phase | | (inventive) | (comparative) |
| A- | Tridecyl trimellitate | 4.4 | 4.4 |
| | Vinylpyrrolidone/1-hexa-decene | 1.6 | 1.6 |
| | copolymer sold or manufactured | | |
| | by ISP under the reference | | |
| | Antaron V-216 | | |
| | Vinylpyrrolidone/1-eicosene | 0.8 | 0.8 |
| | copolymer sold or manu-factured | | |
| | by ISP under the reference | | |
| | Antaron V-220 | | |
| | Bisdiglyceryl polyacyl-adipate-2 | 1.6 | 1.6 |
| | BHT | 0.04 | 0.04 |
| | Polyglyceryl-2-triisostearate | 3.4 | 3.4 |
| | Cetyl dimethicone copolyol | — | 10 |
| | Polyglyceryl-2 monoiso-stearate | 10 | — |
| | Isononyl isononanoate | 10 | 10 |
| B- | Polyethylene wax (MM = | 6.6 | 6.6 |
| | 500 g/mol) | | |
| | Octacosanyl stearate | 5.5 | 5.5 |
| C- | Red 21 | 0.06 | 0.06 |
| | Red 7 | 0.2 | 0.2 |
| | Iron oxides | 2.2 | 2.2 |
| C′- | Ethylene glycol methacrylate/ | 1 | 1 |
| | lauryl methacrylate copolymer | | |
| | manufactured or sold under the | | |
| | reference Polytrap 603 by | | |
| | Advanced Polymer Systems | | |
| | N-Lauroyl-L-lysine | 2.5 | 2.5 |
| | Kaolin | 5 | 5 |
| D- | Hydrogenated isoparaffin | qs 100 | qs 100 |
| | manufactured or sold under the | | |
| | reference Parléam by Nippon Oil | | |
| | and Fats | | |
| | Hydrogenated polyisobutene | 8.5 | 8.5 |
| | manufactured or sold under the | | |
| | reference Panalane H-300E by | | |
| | Amoco Chemical | | |
| | Phenyltrimethicone sold or | 12.7 | 12.7 |
| | manufactured by Dow Corning | | |
| | under the reference DC 556 | | |
| | Pyrogenic silica | 3 | 3 |
| E- | Titanium oxide mica | 1.8 | 1.8 |

The procedure was the same as that of Examples 1 and 2 above.

Cosmetic Evaluation

The staying power, migration, gloss and comfort were evaluated by the same methods as those of Examples 1 and 2.

The composition of Example 3 was adjudged to be glossy, comfortable and of low migration and possesses a staying power which is markedly greater than that of the composition of Example 4 (the staying power after testing was evaluated at 71 for composition 3 as against 49 for the composition of Example 4).

What is claimed is:

1. An anhydrous care or makeup composition comprising, in a cosmetically acceptable medium,
at least one non-volatile silicone compound,
at least one non-silicone fat-soluble polymer and
at least one hydrocarbon dispersant which has solubility parameters δd and δa meeting the following conditions: 16.2≦δ_{d}≦20 (J/cm³)^{1/2} and 9.1≦δₐ≦20 (J/cm³)^{1/2}, wherein the at least one hydrocarbon dispersant comprises carbon and hydrogen atoms and at least one functional group chosen from hydroxyl, ester, ether, carboxylic acid and amide functional groups; and
wherein the composition is anhydrous.

2. The composition according to claim 1, wherein the composition comprises less than 10% by weight of at least one volatile oil relative to the total weight of the composition.

3. The composition according to claim 2, wherein the composition comprises less than 5% by weight of at least one volatile oil relative to the total weight of the composition.

4. The composition according to claim 3, wherein the composition comprises less than 2% by weight of at least one volatile oil relative to the total weight of the composition.

5. The composition according to claim 1, wherein the at least one hydrocarbon dispersant has the solubility parameters δd and δa which meet the following conditions: 16.3≦δ_{d}≦19 (J/cm³)^{1/2} and 10≦δₐ≦18.1 (J/cm³)^{1/2}.

6. The composition according to claim 5, wherein the at least one hydrocarbon dispersant has the solubility parameters δd and δa which meet the following conditions: 16.9≦δ_{d}≦18 (J/cm³)^{1/2} and 13≦δₐ≦14.5 (J/cm³)^{1/2}.

7. The composition according to claim 1, wherein the at least one hydrocarbon dispersant has at least one characteristic chosen from characteristics of being fluid at room temperature (25° C.) and having a refractive index ≧1.45 at 20° C.

8. The composition according to claim 1, wherein the at least one hydrocarbon dispersant has a chemical structure which comprises at least one polar group chosen from —COOH; —OH; ethylene oxide —(O—CH₂—CH₂—); propylene oxide 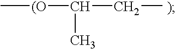
—PO₄; NHR; NR₁R₂, wherein R₁ and R₂, which may be identical or different, optionally form a ring and are each chosen from linear and branched C₁ to C₂₀ alkyl and alkoxy radicals, and 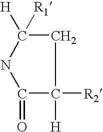
wherein R₁′ and R₂′, which may be identical or different, are each chosen from H and linear and branched C₁ to C₂₀ alkyl and alkoxy radicals.

9. The composition according to claim 1, wherein the at least one hydrocarbon dispersant is chosen from:
(a) ether-modified fatty alcohols,
(b) esters resulting from the reaction of at least one fatty acid with at least one addition product chosen from addition products of ethylene oxide and glycerol and addition products of ethylene oxide and polyglycerol,
(c) esters resulting from the reaction of at least one compound chosen from glycerol and polyglycerol with at least one addition product of ethylene oxide and a fatty acid chosen from saturated and unsaturated fatty acids,
(d) partial esters resulting from the reaction of at least one compound chosen from saturated and unsaturated, linear and branched fatty acids, ricinoleic acid, and 12-hydroxystearic acid with at least one polyol,
(e) esters resulting from the reaction of sorbitan with at least one fatty acid chosen from saturated and unsaturated, linear and branched fatty acids,
(f) ether-modified sorbitan esters,
(g) addition products of ethylene oxide with at least one compound chosen from castor oil and hydrogenated castor oil, and
(h) trialkyl phosphates and alkyl mono-, di- and triphosphates.

10. The composition according to claim 9, wherein, in (a), the ether-modified fatty alcohols are chosen from the addition products of at least one compound chosen from ethylene oxide and propylene oxide with at least one other compound chosen from linear and branched fatty alcohols and alkylphenols.

11. The composition according to claim 9, wherein, in (d), the at least one polyol is chosen from glycerol, polyglycerol, pentaerythritol, and saccharide alcohols.

12. The composition according to claim 11, wherein the saccharide alcohols are sorbitols.

13. The composition according to claim 11, wherein the partial esters are chosen from esters of polyglycerol.

14. The composition according to claim 9, wherein, in (f), the ether-modified sorbitan esters are chosen from esters resulting from at least one reaction chosen from the reaction of sorbitan with at least one addition product of ethylene oxide and a fatty acid chosen from saturated and unsaturated fatty acids and the reaction of at least one fatty acid chosen from saturated and unsaturated fatty acids with at least one addition product of ethylene oxide and sorbitan.

15. The composition according to claim 1, wherein the at least one hydrocarbon dispersant is chosen from monoesters, diesters and esters resulting from a partial esterification.

16. The composition according to claim 1, wherein the at least one hydrocarbon dispersant is chosen from:
(a) addition products of at least one compound chosen from ranging from 2 to 30 moles of ethylene oxide and ranging from 0 to 5 moles of propylene oxide with at least one other compound chosen from linear and branched C₈ to C₄₀ fatty alcohols and alkylphenols,
(b) esters resulting from the reaction of at least one fatty acid chosen from C₈ to C₄₀ fatty acids with at least one addition product chosen from addition products of from 1 to 30 moles of ethylene oxide and glycerol and addition products of from 1 to 30 moles of ethylene oxide and polyglycerol,
(c) esters resulting from the reaction of at least one compound chosen from glycerol and polyglycerol with at least one addition product of from 2 to 30 moles of ethylene oxide and a fatty acid chosen from C₈ to C₄₀ saturated and unsaturated fatty acids,
(d) partial esters resulting from the reaction of at least one compound chosen from saturated and unsaturated C₈ to C₄₀ linear and branched fatty acids, ricinoleic acid, and 12-hydroxystearic acid with at least one other compound chosen from glycerol, polyglycerol, pentaerythritol and sorbitol,
(e) esters resulting from the reaction of sorbitan with at least one fatty acid chosen from saturated and unsaturated C₈ to C₄₀ linear and branched fatty acids,
(f) esters resulting from at least one reaction chosen from the reaction of sorbitan with at least one addition product of from 2 to 30 moles of ethylene oxide and at least one fatty acid chosen from saturated and unsaturated C₈ to C₄₀ fatty acids and the reaction of at least one fatty acid chosen from C₈ to C₄₀ saturated and unsaturated fatty acids with at least one addition product of from 2 to 30 moles of ethylene oxide and sorbitan,
(g) addition products of from 2 to 60 moles of ethylene oxide with at least one compound chosen from castor oil and hydrogenated castor oil, and
(h) trialkyl phosphates and alkyl mono-, di- and triphosphates.

17. The composition according to claim 16, wherein, in (a), the fatty alcohols are chosen from linear and branched C₈ to C₂₂ fatty alcohols.

18. The composition according to claim 16, wherein, in (b), the at least one fatty acid is chosen from C₈ to C₂₂ fatty acids.

19. The composition according to claim 16, wherein, in (c), the fatty acid is chosen from C₈ to C₂₂ saturated and unsaturated fatty acids.

20. The composition according to claim 16, wherein, in (d), the at least one compound is chosen from saturated and unsaturated C₈ to C₂₂ linear and branched fatty acids.

21. The composition according to claim 16, wherein, in (e), the at least one fatty acid is chosen from saturated and unsaturated C₈ to C₂₂ linear and branched fatty acids,

22. The composition according to claim 16, wherein, in (f), the at least one fatty acid is chosen from saturated and unsaturated C₈ to C₂₂ fatty acids.

23. The composition according to claim 1, wherein the at least one hydrocarbon dispersant is chosen from ethoxylated myristyl alcohol comprising 15 ethylene oxide groups, ethoxylated polyglyceryl-2 monoisostearate comprising 5 ethylene oxide groups, polyglyceryl-3 diisostearate, glyceryl monoisostearate, polyglyceryl-2 monoisostearate, polyglyceryl-3 isostearate, polyglyceryl-4 isostearate, polyglyceryl-6 monoisostearate, polyglyceryl-10 monoisostearate, polyglyceryl-2 monooleate, sorbitan isostearate, sorbitan monooleate, and ethoxylated sorbitan monooleate comprising 5 ethylene oxide groups.

24. The composition according to claim 1, wherein the at least one hydrocarbon dispersant is chosen from partial esters of polyglycerol and isostearic acid, partial esters of polyglycerol and oleic acid, and partial esters of sorbitan and oleic acid.

25. The composition according to claim 1, wherein the at least one hydrocarbon dispersant is chosen from polyglyceryl-3 diisostearate, polyglyceryl-2 monoisostearate, polyglyceryl-2 monooleate, and sorbitan monooleate.

26. The composition according to claim 1, wherein the at least one hydrocarbon dispersant is in a concentration ranging from 0.5 to 40% by weight of the total weight of the composition.

27. The composition according to claim 26, wherein the at least one hydrocarbon dispersant is in a concentration ranging from 3 to 20% by weight of the total weight of the composition.

28. The composition according to claim 27, wherein the at least one hydrocarbon dispersant is in a concentration ranging from 5 to 15% by weight of the total weight of the composition.

29. The composition according to claim 1, wherein the at least one non-volatile silicone compound has a viscosity ranging from 5 to 1 000 000 cSt at 25° C.

30. The composition according to claim 29, wherein the at least one non-volatile silicone compound has a viscosity ranging from 10 to 500 000 cSt at 25° C.

31. The composition according to claim 30, wherein the at least one non-volatile silicone compound has a viscosity ranging from 10 to 5 000 cSt at 25° C.

32. The composition according to claim 1, wherein the at least one non-volatile silicone compound is chosen from non-volatile polydimethylsiloxanes; polydimethylsiloxanes comprising at least one group chosen from alkyl, alkoxy and phenyl groups, pendently or at the silicone chain end, wherein the at least one group comprises from 2 to 24 carbon atoms; phenyltrimethicones, phenyldimethicones, phenyltrimethylsiloxydiphenylsiloxanes, diphenyldimethicones, diphenylmethyldiphenyltrisiloxanes and 2-phenylethyl trimethylsiloxysilicates; fluorinated silicones comprising at least one fluorinated group pendently or at the silicone chain end, comprising from 1 to 12 carbon atoms, some or all of the hydrogens thereof being substituted by fluorine atoms; silicone resins; and silicone gums.

33. The composition according to claim 1, wherein the at least one non-volatile silicone compound is in a concentration ranging from 0.5 to 90% by weight of the total weight of the composition.

34. The composition according to claim 33, wherein the at least one non-volatile silicone compound is in a concentration ranging from 5 to 60% by weight of the total weight of the composition.

35. The composition according to claim 34, wherein the at least one non-volatile silicone compound is in a concentration ranging from 10 to 50% by weight of the total weight of the composition.

36. The composition according to claim 1, wherein the at least one non-silicone fat-soluble polymer has a molecular mass ranging from 400 to 500 000 g/mol.

37. The composition according to claim 36, wherein the at least one non-silicone fat-soluble polymer has a molecular mass ranging from 500 to 100 000 g/mol.

38. The composition according to claim 1, wherein the at least one non-silicone fat-soluble polymer is chosen from vinyl polymers, fat-soluble polyethers, and non-crosslinked polyesters.

39. The composition according to claim 1, wherein the at least one non-silicone fat-soluble polymer is chosen from:
(a) branched non-crystalline olefin homopolymers, non-branched olefin copolymers, non-crystalline hydrogenated diene homopolymers and copolymers, linear and branched oligomers, alkyl (meth)acrylate homopolymers and copolymers, perfluorinated alkyl (meth)acrylate oligomers, homopolymers and copolymers having a molar mass of less than or equal to 10 000 g/mol, vinyl ester oligomers, homopolymers and copolymers having at least one alkyl group chosen from C₈-C₃₀ alkyl groups and a molar mass of less than or equal to 10 000 g/mol, vinyl ether oligomers, homopolymers and copolymers having at least one alkyl group chosen from C₈-C₃₀ alkyl groups and a molar mass of less than or equal to 10 000 g/mol,
(b) fat-soluble polyethers resulting from the polyetherification of at least one diol chosen from C₂-C₁₀₀ diols, and
(c) non-crosslinked polyesters resulting from the polycondensation of an acid chosen from C₄-C₅₀ linear and branched dicarboxylic and polycarboxylic acids and a compound chosen from C₂-C₅₀ diols and polyols.

40. The composition according to claim 39, wherein, in (a), the alkyl (meth)acrylate homopolymers and copolymers have a C₈-C₃₀ alkyl group and a molar mass of less than or equal to 10 000 g/mol.

41. The composition according to claim 39, wherein, in (a), the perfluorinated alkyl (meth)acrylate oligomers, homopolymers and copolymers have a molar mass of less than or equal to 8 000 g/mol.

42. The composition according to claim 39, wherein, in (a), the vinyl ester oligomers, homopolymers and copolymers have a molar mass of less than or equal to 8 000 g/mol.

43. The composition according to claim 39, wherein, in (a), the vinyl ether oligomers, homopolymers and copolymers have a molar mass of less than or equal to 8 000 g/mol.

44. The composition according to claim 39, wherein, in (b), the at least one diol is chosen from C₂-C₅₀ diols.

45. The composition according to claim 1, wherein the at least one fat-soluble polymer is chosen from polybutylenes, hydrogenated polyisobutylenes, polydecenes, hydrogenated poloydecenes, vinylpyrrolidone (VP)/olefin copolymers having a number of carbons ranging from 8 to 30, hydrogenated polybutadiene, hydrogenated polyisoprene, polyvinyl laurate, vinyl acetate/allyl stearate copolymer, and PEG-45/dodecyl glycol copolymer.

46. The composition according to claim 45, wherein the vinylpyrrolidone (VP)/olefin copolymers have a number of carbons ranging from 10 to 30.

47. The composition according to claim 1, wherein the at least one fat-soluble polymer is in a concentration ranging from 1 to 98.5% by weight of the total weight of the composition.

48. The composition according to claim 47, wherein the at least one fat-soluble polymer is in a concentration ranging from 2 to 85% by weight of the total weight of the composition.

49. The composition according to claim 48, wherein the at least one fat-soluble polymer is in a concentration ranging from 5 to 70% by weight of the total weight of the composition.

50. The composition according to claim 49, wherein the at least one fat-soluble polymer is in a concentration ranging from 5 to 60% by weight of the total weight of the composition.

51. The composition according to claim 1, further comprising at least one colorant.

52. The composition according to claim 51, wherein the at least one colorant is chosen from dyes which are soluble or dispersible in the composition, pigments, and nacres.

53. The composition according to claim 51, wherein the at least one colorant is in a concentration ranging from 0.5 to 50% by weight relative to the total weight of the composition.

54. The composition according to claim 53, wherein the at least one colorant is in a concentration ranging from 2 to 40% by weight relative to the total weight of the composition.

55. The composition according to claim 54, wherein the at least one colorant is in a concentration ranging from 5 to 30% by weight relative to the total weight of the composition.

56. The composition according to claim 1, comprising a particulate phase.

57. The composition according to claim 1, further comprising at least one additional non-aqueous compound other than the at least one non-volatile silicone compound, the at least one non-silicone fat-soluble polymer and the at least one hydrocarbon dispersant, wherein the at least one additional non-aqueous compound is chosen from oils, fatty substances which are pastelike at room temperature, waxes, gums, and resins.

58. The composition according to claim 1, further comprising at least one wax.

59. The composition according to claim 58, wherein the at least one wax is in a concentration ranging from 0.01 to 50% by weight of the total weight of the composition.

60. The composition according to claim 59, wherein the at least one wax is in a concentration ranging from 2 to 40% by weight of the total weight of the composition.

61. The composition according to claim 60, wherein the at least one wax is in a concentration ranging from 5 to 30% by weight of the total weight of the composition.

62. The composition according to claim 1, further comprising at least one additive chosen from antioxidants, preservatives, neutralizing agents, lipophilic gelling agents and liquid non-aqueous compounds, dispersants, and active cosmetic substances.

63. The composition according to claim 62, comprising at least one active cosmetic substance chosen from vitamins (A, E, C, B₃, F), provitamins, active soothing agents, plant extracts and essential oils, protective agents and restructuring agents, active freshness agents, emollients, moisturizers, active anti-wrinkle agents, and essential fatty acids.

64. The composition according to claim 1, wherein the composition is provided in the form of a cast or compacted product.

65. The composition according to claim 64, wherein the composition is provided in the form of a lipstick or lip gloss.

66. A cosmetic method of imparting to a film of anhydrous cosmetic composition at least one property chosen from properties of colour fastness, gloss, comfort and non-migration comprising introducing into the composition at least one non-volatile silicone compound, at least one non-silicone fat-soluble polymer and at least one hydrocarbon dispersant which has solubility parameters δd and δa meeting the following conditions: 16.2≦δ_{d}≦20 (J/cm³)^{1/2} and 90.1≦δₐ≦20 (J/cm³)^{1/2}, wherein the at least one hydrocarbon dispersant comprises carbon and hydrogen atoms and at least one functional group chosen from hydroxyl, ester, ether, carboxylic acid and amide functional groups.

67. The method according to claim 66, wherein the at least one hydrocarbon dispersant is chosen from:
(a) ether-modified fatty alcohols,
(b) esters resulting from the reaction of at least one fatty acid with at least one addition product chosen from addition products of ethylene oxide and glycerol and addition products of ethylene oxide and polyglycerol,
(c) esters resulting from the reaction of at least one compound chosen from glycerol and polyglycerol with at least one addition product of ethylene oxide and a fatty acid chosen from saturated and unsaturated fatty acids,
(d) partial esters resulting from the reaction of at least one compound chosen from saturated and unsaturated, linear and branched fatty acids, ricinoleic acid, and 12-hydroxystearic acid with at least one polyol,
(e) esters resulting from the reaction of sorbitan with at least one fatty acid chosen from saturated and unsaturated, linear and branched fatty acids,
(f) ether-modified sorbitan esters,
(g) addition products of ethylene oxide with at least one compound chosen from castor oil and hydrogenated castor oil, and
(h) trialkyl phosphates and alkyl mono-, di- and triphosphates.

68. The method according to claim 67, wherein, in (a), the ether-modified fatty alcohols are chosen from the addition products of at least one compound chosen from ethylene oxide and propylene oxide with at least one other compound chosen from linear and branched fatty alcohols and alkylphenols.

69. The method according to claim 67, wherein, in (b), the esters are chosen from monoesters and diesters.

70. The method according to claim 67, wherein, in (d), the at least one polyol is chosen from glycerol, polyglycerol, pentaerythritol, saccharide alcohols.

71. The method according to claim 70, wherein the saccharide alcohols are sorbitols.

72. The method according to claim 70, wherein the partial esters are chosen from esters of polyglycerol,

73. The method according to claim 67, wherein, in (f), the ether-modified sorbitan esters are chosen from esters resulting from at least one reaction chosen from the reaction of sorbitan with at least one addition product of ethylene oxide and a fatty acid chosen from saturated and unsaturated fatty acids and the reaction of at least one fatty acid chosen from saturated and unsaturated fatty acids with at least one addition product of ethylene oxide and sorbitan.

74. The method according to claim 66, wherein the at least one non-volatile silicone compound is chosen from non-volatile polydimethylsiloxanes; polydimethylsiloxanes comprising at least one group chosen from alkyl, alkoxy and phenyl groups, pendently or at the silicone chain end, wherein the at least one group comprises from 2 to 24 carbon atoms; phenyltrimethicones, phenyldimethicones, phenyltrimethylsiloxydiphenylsiloxanes, diphenyldimethicones, diphenylmethyldiphenyltrisiloxanes and 2-phenylethyl trimethylsiloxysilicates; fluorinated silicones comprising at least one fluorinated group pendently or at the silicone chain end, comprising from 1 to 12 carbon atoms, some or all of the hydrogens thereof being substituted by fluorine atoms; silicone resins, and silicone gums.

75. The method according to claim 66, wherein the at least one non-silicone fat-soluble polymer is chosen from:
(a) branched non-crystalline olefin homopolymers, non-branched olefin copolymers, non-crystalline hydrogenated diene homopolymers and copolymers, linear and branched oligomers, alkyl (meth)acrylate homopolymers and copolymers, perfluorinated alkyl (meth)acrylate oligomers, homopolymers and copolymers having a molar mass of less than or equal to 10 000 g/mol, vinyl ester oligomers, homopolymers and copolymers having at least one alkyl group chosen from C₈-C₃₀ alkyl groups and a molar mass of less than or equal to 10 000 g/mol, vinyl ether oligomers, homopolymers and copolymers having at least one alkyl group chosen from C₈-C₃₀ alkyl groups and a molar mass of less than or equal to 10 000 g/mol,
(b) fat-soluble polyethers resulting from the polyetherification of at least one diol chosen from C₂-C₁₀₀ diols, and
(c) non-crosslinked polyesters resulting from the polycondensation of an acid chosen from C₄-C₅₀ linear and branched dicarboxylic and polycarboxylic acids and a compound chosen from C₂-C₅₀ diols and polyols.

76. The method according to claim 75, wherein, in (a), the alkyl (meth)acrylate homopolymers and copolymers have a C₈-C₃₀ alkyl group and a molar mass of less than or equal to 10 000 g/mol.

77. The method according to claim 75, wherein, in (a), the perfluorinated alkyl (meth)acrylate oligomers, homopolymers and copolymers have a molar mass of less than or equal to 8 000 g/mol.

78. The method according to claim 75, wherein, in (a), the vinyl ester oligomers, homopolymers and copolymers have a molar mass of less than or equal to 8 000 g/mol.

79. The method according to claim 75, wherein, in (a), the vinyl ether oligomers, homopolymers and copolymers have a molar mass of less than or equal to 8 000 g/mol.

80. The method according to claim 75, wherein, in (b), the at least one diol is chosen from C₂-C₅₀ diols.

81. A method of making an anhydrous cosmetic composition comprising including in the composition at least one non-volatile silicone compound, at least one non-silicone fat-soluble polymer and at least one hydrocarbon dispersant which has solubility parameters δd and δa meeting the following conditions: 16.2≦δ_{d}≦20 (J/cm³)^{1/2} and 90.1≦δₐ≦20 (J/cm³)^{1/2}, wherein the composition has enhanced staying power, and has at least one characteristic chosen from characteristics of being glossy, comfortable and non-migrating, and the at least one hydrocarbon dispersant comprises carbon and hydrogen atoms and at least one functional group chosen from hydroxyl, ester, ether, carboxylic acid and amide functional groups.

82. The method according to claim 81, wherein the composition has good colour fastness.

83. An agent for imparting to an anhydrous cosmetic composition at least one property chosen from properties of staying power, gloss, comfort and non-migration comprising at least one non-volatile silicone compound, at least one non-silicone fat-soluble polymer and at least one hydrocarbon dispersant which has solubility parameters δd and δa meeting the following conditions: 16.2≦δ_{d}≦20 (J/cm³)^{1/2} and 9.1≦δₐ≦20 (J/cm³)^{1/2}, wherein the at least one hydrocarbon dispersant comprises carbon and hydrogen atoms and at least one functional group chosen from hydroxyl, ester, ether, carboxylic acid and amide functional groups, and the agent is effective in imparting to the anhydrous cosmetic composition the at least one property.

84. The agent according to claim 83, wherein the properties of staying power are properties of colour fastness.

85. The method according to claim 81, wherein the at least one hydrocarbon dispersant is chosen from:
(a) ether-modified fatty alcohols,
(b) esters resulting from the reaction of at least one fatty acid with at least one addition product chosen from addition products of ethylene oxide and glycerol and addition products of ethylene oxide and polyglycerol,
(c) esters resulting from the reaction of at least one compound chosen from glycerol and polyglycerol with at least one addition product of ethylene oxide and a fatty acid chosen from saturated and unsaturated fatty acids,
(d) partial esters resulting from the reaction of at least one compound chosen from saturated and unsaturated, linear and branched fatty acids, ricinoleic acid, and 12-hydroxystearic acid with at least one polyol,
(e) esters resulting from the reaction of sorbitan with at least one fatty acid chosen from saturated and unsaturated, linear and branched fatty acids,
(f) ether-modified sorbitan esters,
(g) addition products of ethylene oxide with at least one compound chosen from castor oil and hydrogenated castor oil, and
(h) trialkyl phosphates and alkyl mono-, di- and triphosphates.

86. The method according to claim 85, wherein, in (a), the ether-modified fatty alcohols are chosen from the addition products of at least one compound chosen from ethylene oxide and propylene oxide with at least one other compound chosen from linear and branched fatty alcohols and alkylphenols.

87. The method according to claim 85, wherein, in (d), the at least one polyol is chosen from glycerol, polyglycerol, pentaerythritol, and saccharide alcohols.

88. The method according to claim 87, wherein the saccharide alcohols are sorbitols.

89. The method according to claim 87, wherein the partial esters are chosen from esters of polyglycerol.

90. The method according to claim 85, wherein, in (f), the ether-modified sorbitan esters are chosen from esters resulting from at least one reaction chosen from the reaction of sorbitan with at least one addition product of ethylene oxide and a fatty acid chosen from saturated and unsaturated fatty acids and the reaction of at least one fatty acid chosen from saturated and unsaturated fatty acids with at least one addition product of ethylene oxide and sorbitan.

91. The agent according to claim 83, wherein the at least one hydrocarbon dispersant is chosen from:
(a) ether-modified fatty alcohols,
(b) esters resulting from the reaction of at least one fatty acid with at least one addition product chosen from addition products of ethylene oxide and glycerol and addition products of ethylene oxide and polyglycerol,
(c) esters resulting from the reaction of at least one compound chosen from glycerol and polyglycerol with at least one addition product of ethylene oxide and a fatty acid chosen from saturated and unsaturated fatty acids,
(d) partial esters resulting from the reaction of at least one compound chosen from saturated and unsaturated, linear and branched fatty acids, ricinoleic acid, and 12-hydroxystearic acid with at least one polyol,
(e) esters resulting from the reaction of sorbitan with at least one fatty acid chosen from saturated and unsaturated, linear and branched fatty acids,
(f) ether-modified sorbitan esters,
(g) addition products of ethylene oxide with at least one compound chosen from castor oil and hydrogenated castor oil, and
(h) trialkyl phosphates and alkyl mono-, di- and triphosphates.

92. The agent according to claim 91, wherein, in (a), the ether-modified fatty alcohols are chosen from the addition products of at least one compound chosen from ethylene oxide and propylene oxide with at least one other compound chosen from linear and branched fatty alcohols and alkylphenols.

93. The agent according to claim 91, wherein, in (d), the at least one polyol is chosen from glycerol, polyglycerol, pentaerythritol, and saccharide alcohols.

94. The agent according to claim 93, wherein the saccharide alcohols are sorbitols.

95. The agent according to claim 93, wherein the partial esters are chosen from esters of polyglycerol.

96. The agent according to claim 91, wherein, in (f), the ether-modified sorbitan esters are chosen from esters resulting from at least one reaction chosen from the reaction of sorbitan with at least one addition product of ethylene oxide and a fatty acid chosen from saturated and unsaturated fatty acids and the reaction of at least one fatty acid chosen from saturated and unsaturated fatty acids with at least one addition product of ethylene oxide and sorbitan.

97. The method according to claim 81, wherein the at least one non-volatile silicone compound is chosen from non-volatile polydimethylsiloxanes; polydimethylsiloxanes comprising at least one group chosen from alkyl, alkoxy and phenyl groups, pendently or at the silicone chain end, wherein the at least one group comprises from 2 to 24 carbon atoms; phenyltrimethicones, phenyldimethicones, phenyltrimethylsiloxydiphenylsiloxanes, diphenyldimethicones, diphenylmethyldiphenyltrisiloxanes and 2-phenylethyl trimethylsiloxysilicates; fluorinated silicones comprising at least one fluorinated group pendently or at the silicone chain end, comprising from 1 to 12 carbon atoms, some or all of the hydrogens thereof being substituted by fluorine atoms; silicone resins; and silicone gums.

98. The agent according to claim 83, wherein the at least one non-volatile silicone compound is chosen from non-volatile polydimethylsiloxanes; polydimethylsiloxanes comprising at least one group chosen from alkyl, alkoxy and phenyl groups, pendently or at the silicone chain end, wherein the at least one group comprises from 2 to 24 carbon atoms; phenyltrimethicones, phenyldimethicones, phenyltrimethylsiloxydiphenylsiloxanes, diphenyldimethicones, diphenylmethyldiphenyltrisiloxanes and 2-phenylethyl trimethylsiloxysilicates; fluorinated silicones comprising at least one fluorinated group pendently or at the silicone chain end, comprising from 1 to 12 carbon atoms, some or all of the hydrogens thereof being substituted by fluorine atoms; silicone resins; and silicone gums.

99. The method according to claim 81, wherein the at least one non-silicone fat-soluble polymer is chosen from:
(a) branched non-crystalline olefin homopolymers, non-branched olefin copolymers, non-crystalline hydrogenated diene homopolymers and copolymers, linear and branched oligomers, alkyl (meth)acrylate homopolymers and copolymers, perfluorinated alkyl (meth)acrylate oligomers, homopolymers and copolymers having a molar mass of less than or equal to 10 000 g/mol, vinyl ester oligomers, homopolymers and copolymers having at least one alkyl group chosen from C₈-C₃₀ alkyl groups and a molar mass of less than or equal to 10 000 g/mol, vinyl ether oligomers, homopolymers and copolymers having at least one alkyl group chosen from C₈-C₃₀ alkyl groups and a molar mass of less than or equal to 10 000 g/mol,
(b) fat-soluble polyethers resulting from the polyetherification of at least one diol chosen from C₂-C₁₀₀ diols, and
(c) non-crosslinked polyesters resulting from the polycondensation of an acid chosen from C₄-C₅₀ linear and branched dicarboxylic and polycarboxylic acids and a compound chosen from C₂-C₅₀ diols and polyols.

100. The method according to claim 99, wherein, in (a), the alkyl (meth)acrylate homopolymers and copolymers have a C₈-C₃₀ alkyl group and a molar mass of less than or equal to 10 000 g/mol.

101. The method according to claim 99, wherein, in (a), the perfluorinated alkyl (meth)acrylate oligomers, homopolymers and copolymers have a molar mass of less than or equal to 8 000 g/mol.

102. The method according to claim 99, wherein, in (a), the vinyl ester oligomers, homopolymers and copolymers have a molar mass of less than or equal to 8 000 g/mol.

103. The method according to claim 99, wherein, in (a), the vinyl ether oligomers, homopolymers and copolymers have a molar mass of less than or equal to 8 000 g/mol.

104. The method according to claim 99, wherein, in (b), the at least one diol is chosen from C₂-C₅₀ diols.

105. The agent according to claim 83, wherein the at least one non-silicone fat-soluble polymer is chosen from:
(a) branched non-crystalline olefin homopolymers, non-branched olefin copolymers, non-crystalline hydrogenated diene homopolymers and copolymers, linear and branched oligomers, alkyl (meth)acrylate homopolymers and copolymers, perfluorinated alkyl (meth)acrylate oligomers, homopolymers and copolymers having a molar mass of less than or equal to 10 000 g/mol, vinyl ester oligomers, homopolymers and copolymers having at least one alkyl group chosen from C₈-C₃₀ alkyl groups and a molar mass of less than or equal to 10 000 g/mol, vinyl ether oligomers, homopolymers and copolymers having at least one alkyl group chosen from C₈-C₃₀ alkyl groups and a molar mass of less than or equal to 10 000 g/mol,
(b) fat-soluble polyethers resulting from the polyetherification of at least one diol chosen from C₂-C₁₀₀ diols, and
(c) non-crosslinked polyesters resulting from the polycondensation of an acid chosen from C₄-C₅₀ linear and branched dicarboxylic and polycarboxylic acids and a compound chosen from C₂-C₅₀ diols and polyols.

106. The agent according to claim 105, wherein, in (a), the alkyl (meth)acrylate homopolymers and copolymers have a C₈-C₃₀ alkyl group and a molar mass of less than or equal to 10 000 g/mol.

107. The agent according to claim 105, wherein, in (a), the perfluorinated alkyl (meth)acrylate oligomers, homopolymers and copolymers have a molar mass of less than or equal to 8 000 g/mol.

108. The agent according to claim 105, wherein, in (a), the vinyl ester oligomers, homopolymers and copolymers have a molar mass of less than or equal to 8 000 g/mol.

109. The agent according to claim 105, wherein, in (a), the vinyl ether oligomers, homopolymers and copolymers have a molar mass of less than or equal to 8 000 g/mol.

110. The agent according to claim 105, wherein, in (b), the at least one diol is chosen from C₂-C₅₀ diols.
